(19) United States
(12) Patent Application Publication
(10) Pub. No.: US 2005/0277797 A1
(43) Pub. Date: Dec. 15, 2005

(54) Process for producing hydrocarbons

(75) Inventors: Toshihide Maeda, Nagoya-shi (JP); Kouji Ohya, Nagoya-shi (JP)

Correspondence Address: KODA & ANDROLIA, 2029 CENTURY PARK EAST, SUITE 1140, LOS ANGELES, CA, 90067, US

(73) Assignee: Daido Steel Co., Ltd.

(21) Appl. No.: 11/150,530

(22) Filed: Jun. 10, 2005

(30) Foreign Application Priority Data
Jun. 10, 2004 (JP) 2004-172573

(51) Int. Cl. C07C 4/04; C10G 9/16

(52) U.S. Cl. 585/648; 585/650; 585/652; 208/132; 585/950

(57) ABSTRACT

The process for producing hydrocarbons of the present invention comprises a thermal cracking process for heating a furnace tube composed of a multi-layered heat resistant metal tube comprising a substrate tube composed of a heat resistant metal and a surface layer of a Cr—Ni alloy formed on an inner surface of the substrate tube to a predetermined temperature, and supplying a hydrocarbon raw material gas into the furnace tube to thermally decompose the hydrocarbon raw material gas; a decoking process for removing coke accumulated on the inner surface of the furnace tube; and a furnace tube replacement process for performing replacement of the furnace tube after the lapse of an operation time longer than the life of the substrate tube in the case of performing the thermal cracking under the same condition by use of a furnace tube composed of only the substrate tube.

### BACKGROUND OF THE INVENTION

1. Field of the Invention

The present invention relates to a process for producing hydrocarbons, more specifically, the process for producing hydrocarbons, in which a hydrocarbon raw material gas such as naphtha and steam are supplied into a furnace tube heated to a high temperature in order to thermally decompose the hydrocarbon raw material gas, thereby producing ethylene, propylene or the like.

2. Description of Related Art

Hydrocarbons such as ethylene and propylene are conventionally produced by thermally decomposing a hydrocarbon raw material gas such as naphtha. In such a thermal cracking of hydrocarbon raw material gas, in general, a thermal cracking furnace comprising a furnace tube having a total length of about 100 m to 1000 m and a heating means for heating the furnace tube from the outside is used.

The thermal cracking of hydrocarbon raw material gas using the thermal cracking furnace is generally carried out according to the following procedure. The hydrocarbon raw material gas and steam are first supplied, at high speed, into the furnace tube heated to a predetermined temperature. When the hydrocarbon raw material gas and steam are supplied into the heated furnace tube, the hydrocarbon raw material gas is thermally decomposed by the steam to generate a decomposed gas including an intended hydrocarbon such as ethylene or propylene.

On the other hand, in accordance with the thermal cracking of the hydrocarbon raw material gas, carbon (coke) is generated as a byproduct and it is accumulated on the inner surface of the furnace tube. The accumulation of coke on the inner surface of the furnace tube causes a reduction in the heat conductivity of the furnace tube. Therefore, in the case that the heat input from the heating means is constant, the internal temperature of the furnace tube gradually lowers in accordance with the accumulation of coke. The composition of the decomposed gas mainly depends on the decomposition temperature (the inner temperature of the furnace tube), therefore, keeping the decomposition temperature constant is needed for production of an intended hydrocarbon in a constant yield. In general, the decomposition temperature is kept constant by controlling the surface temperature of the furnace tube (tube internal temperature (TMT)), the coil outlet temperature (COT), and the raw material input per unit time (hereinafter referred to simply as “raw material input”), and the like.

When the thermal cracking furnace is continuously operated for a fixed period in a predetermined condition, the layer of coke accumulated on the inner surface of the furnace tube is gradually thickened, in accordance with this, the pressure loss of the furnace tube is also increased. When the pressure loss becomes unignorable, the operation is interrupted in order to perform the removal of the accumulated coke from the inner surface of the furnace tube (decoking). The decoking is carried out, in general, by supplying only steam into the furnace tube to burn off the coke.

The thermal cracking of the hydrocarbon raw material gas is performed by repeating the continuous operation for a fixed period and the decoking as is described above. The furnace tube is exposed to a high temperature with the coke accumulated on the inner surface. Therefore, the carbon is diffused to the internal part of the furnace tube to gradually deteriorate it with the lapse of time. Accordingly, the operation is stopped after the lapse of a certain fixed operation time in order to perform replacement of the furnace tube.

To raise the yield of expensive ethylene and propylene in ethylene cracking furnaces is a longstanding subject. In order to raise the yield of ethylene, various devices were made in the past mainly for raising the decomposition temperature. Therefore, a heat resistant centrifugal casting steel pipe with high-temperature strength has been increasingly used instead of a stainless steel pipe (e.g., SUS 304, SUS 310, etc.) used in the past. Consequently, the decomposition temperature has been raised from 760-780° C. to 800-920° C.

Developments and efforts for increasing the yield have been made so far, however, they are nearly getting close to the limit with respect to material of the furnace tube. Therefore, attempts to raise the decomposition temperature by use of the furnace tubes composed of conventional materials result in rapid progress of carburization to the furnace tubes, which might cause a shorted life of the furnace tube such as breakage by embrittlement. On the other hand, applications of oxide dispersion strengthened alloy (ODS alloy) such as TD-Ni alloy or TD-Ni—Cr alloy, and ceramics are also examined in order to further raise the decomposition temperature, however, industrial use of these materials is considered to be difficult because they are very expensive.

To solve this problem, in Patent Reference 1, a multi-layered heat resistant metal tube comprising a built-up layer of a Cr—Ni—Mo-based alloy, which is formed on the inner surface and/or outer surface of the heat resistant metal, is proposed by the present applicant. In Patent Reference 2, a multi-layered heat resistant metal tube comprising a built-up layer of a Cr—Ni alloy containing 35 wt % or more of Cr with Ni %≧0.5Cr %, which is formed on an inner surface and/or an outer surface of the heat resistant metal is also disclosed. In Patent References 1 and 2, it is described that use of such multi-layered heat resistant metal tubes as furnace tubes enables improvement in coking resistance.

Patent Reference 1: Japanese Patent Application Laid-Open No. 2001-113389

Patent Reference 2: Japanese Patent Application Laid-Open No. 2003-001427

The multi-layered heat resistant metal tubes disclosed in Patent References 1 and 2 are excellent in heat resistance and coking resistance. On the other hand, the multi-layered heat resistant metal tubes are higher in cost, compared with conventional furnace tubes composed of only heat resistant metals, because they need formation of a surface layer on the inner surface of the furnace tubes by using a method of build-up welding, and the like. Accordingly, in order to bring out the maximum characteristics of the multi-layered heat resistant metal tubes, optimization of operating conditions in thermal cracking furnaces is needed. However, an operating method suitable to such multi-layered heat resistant metal tubes has not been proposed yet.

### SUMMARY OF THE INVENTION

The problem to be solved by the present invention is to significantly reduce the production cost of hydrocarbons by optimizing the operating conditions for thermal cracking of a hydrocarbon raw material gas by use of such a multi-layered heat resistant metal tube.

To solve the abovementioned problem, a process for producing hydrocarbons of the present invention comprises a thermal cracking process for heating a furnace tube composed of a multi-layered heat resistant metal tube comprising a substrate tube composed of a heat resistant metal and a surface layer composed of a Cr—Ni alloy formed on an inner surface of the substrate tube to a predetermined temperature, and supplying a hydrocarbon raw material gas into the furnace tube to thermally decompose the hydrocarbon raw material gas; a decoking process for removing coke accumulated on the inner surface of the furnace tube; and a furnace tube replacement process for replacing the furnace tube after the lapse of an operation time longer than the life of the substrate tube in the case of performing the thermal cracking under the same condition by use of a furnace tube composed of only the substrate tube.

When the multi-layered heat resistant metal tube is used as the furnace tube, the heat resistance and coking resistance of the furnace tube are improved. Therefore, the life of the furnace tube becomes longer than that of a conventional furnace tube composed of only a heat resistant metal.

The use of the multi-layered heat resistant metal tube enables raising the upper limit value of the tube internal temperature without significantly reducing the life of the furnace tube. Consequently, the run-length to decoking can be prolonged more than in the past in the case of the same raw material input. On the other hand, the raw material input can be increased more than in the past in the case of the same run-length to decoking.

### BRIEF DESCRIPTION OF THE DRAWINGS

FIG. 1A is a schematic view showing the relation between run-length and a tube internal temperature (TMT) in the case of a constant raw material input per unit time;

FIG. 1B is a schematic view showing the relation between run-length and a tube internal temperature (TMT) in the case of an increased raw material input per unit time;

FIG. 2 is a schematic view showing the production process of hydrocarbons;

FIG. 3A is a view showing the carbon content of a furnace tube in the case of performing thermal cracking for a fixed period by use of HP—Nb material as the furnace tube;

FIG. 3B is a view showing the carbon content of a furnace tube in the case of performing thermal cracking for a fixed period by use of PTT as the furnace tube;

FIG. 4 is a view showing the relation between the distance from the inner surface of the furnace tube and the hardness of the furnace tube in the case of performing thermal cracking for a fixed period by use of HP—Nb material or PTT as the furnace tube;

FIG. 5 is a view showing the relation between the Larson-Miller parameter (LMP) and the carbon content in a furnace tube in the case of performing thermal cracking for a fixed period by use of HP—Nb material as the furnace tube;

FIG. 6 is a view showing the relation between the Larson-Miller parameter (LMP) and the carbon content in the surface layer in the case of performing thermal cracking for a fixed period by use of PTT as the furnace tube;

FIG. 7A is a view showing the relation of carburization quantity and carbon content in matrix, which are measured for PTT and HP—Nb material; and,

FIG. 7B is a view showing the relation of carburization quantity and Cr content in matrix, which are measured for PTT and HP—Nb material.

### DETAILED DESCRIPTION OF THE PREFERRED EMBODIMENTS

One preferred embodiment of the present invention will be described in detail.

A multi-layered heat resistant metal tube is described first. In the present invention, the multi-layered heat resistant metal tube means one comprising a substrate tube composed of a heat resistant metal and a surface layer composed of a Cr—Ni alloy formed thereon.

The substrate tube is composed of a heat resistant metal having heat resistance capable of enduring the thermal decomposition temperature of a hydrocarbon raw material gas.

The following are examples of practical heat resistant metals constituting the substrate tube:
(1) Iron-based alloy containing 8% or more of Cr (e.g., stainless steel such as SUS 304 or SUS 310);
(2) Heat resistant cast steel (e.g., HK material (25Cr-20Ni), HP material (25Cr-35Ni), HP—Nb material (25Cr-35Ni—Nb), etc.); and
(3) Ni-based superalloy (e.g., Inconel 600H, etc.). Among them, a centrifugal casting steel pipe composed of heat resistant cast steel is suitable as the substrate tube because of high heat resistance and relatively low cost.

The outer diameter and thickness of the substrate tube are not particularly limited, and can be selected in an optimum manner according to the material of the substrate tube, the structure of thermal cracking furnace, the thermal cracking condition, the kind of hydrocarbon raw material gas, and the like. In the case that naphtha is thermally cracked to produce ethylene, propylene or the like, for example, a furnace tube having an inner diameter of about 2 to 4 inches (50.8 to 101.6 mm) and a thickness of about 9 to 11 mm is generally used.

The surface layer is composed of aCr—Ni alloy. Cr—Ni alloy having a predetermined composition is excellent in carburization resistance (coking resistance) in addition to high heat resistance. Therefore, Cr—Ni alloy is suitable as the surface layer for protecting the inside surface of the substrate tube.

Cr is an element necessary for increasing the oxidation resistance of the surface layer and extremely important for increasing the coking resistance. To obtain these effects, 36 wt % or more of Cr is preferable. The more the Cr-content increases, the more both the oxidation resistance and the coking resistance are effective. However, an excessively large content of Cr makes it difficult to stabilize austenitic structure and decreases workability. Thus, 49 wt % or less of Cr is preferable. 40 to 47 wt % of Cr is further preferable.

In such a high temperature as the furnace tube used in an ethylene cracking furnace, Ni has the effect of stably keeping the structure of the furnace tube and improving the coking resistance. To obtain such an effect, 35 wt % or more of Ni is preferable. The more the Ni content increases, the more the structure is stabilized. However, an excessively large content of Ni leads to an increase in cost. Accordingly, 63 wt % or less of Ni is preferable. The Ni content is preferably 0.5 times or more the Cr content, further preferably 1.0 to 1.4 times the Cr content.

A part of Ni can be substituted by Co. Such a substitution further more brings about some improvement in coking resistance. However, Co is more expensive than Ni, use of a large amount of Co increases the cost of the furnace tube. Accordingly, the preferable substitution quantity is 10 wt % or less of Ni, and 5 wt % or more of Ni is further preferable.

The Cr—Ni alloy constituting the surface layer may be composed of only Cr and Ni, but may further include Mo (5.0 wt % or less), B (0.015 wt % or less), Zr (0.015 wt % or less), REM (0.002 wt % or less), Si (1.5 wt % or less), Al (3.0 wt % or less) and the like in addition to Cr and Ni. Proper addition of these elements brings about effects such as improvement in weldability and reduction in crack sensitivity of the deposited metal (refer to Patent Reference 2).

In order to ensure the high coking resistance, it is preferable to restrict impurities contained in the Cr—Ni alloy to a certain fixed amount or less. Elements of which content are to be restricted are, concretely, Fe (10 wt % or less, preferably 5 wt % or less), C (0.1 wt % or less), N (0.3 wt % or less), Mn (1.5 wt % or less), P+S (0.02 wt % or less), O (0.3 wt % or less) and the like (refer to Patent Reference 2).

The thickness of the surface layer is preferably 1.0 mm or more. When the surface layer is formed by build-up welding, impurity elements such as Fe intrude from the heat resistant base metal tube into the built-up layer. A thickness of the built-up layer of less than 1.0 mm should be avoided because a layer containing these impurity elements might be formed on the built-up layer.

In the build-up welding of the surface layer, the larger the thickness of the surface layer is, the smaller the amount of impurity elements is contained in the outermost surface part of the surface layer. Accordingly, the coking-resistance are improved. However, an excessively thickened surface layer is unprofitable and rather leads to an increase in cost. Accordingly, the thickness of the surface layer is preferably set to 5 mm or less. When the surface layer is formed by build-up welding, the thickness of the surface layer is further preferably in the range of 1.5 to 3.0 mm.

A multi-layered heat resistant metal tube with such a surface layer can be produced by use of various methods such as build-up welding, HIP, CIP, explosive welding, diffusion bonding and pressure welding. Among them, plasma transfer arc welding, particularly, plasma powder welding (PPW) which uses filler metals in powder form is suitably adapted. PPW method uses the high-temperature hot plasma as the heat source. Therefore, the substrate surface does not melt deeply in depth, and contamination of the built-up layer by the base material metal can be avoided. Further, powder metal is used as the filler metal, namely, it is not necessary to prepare the filler metal in the form of wires or rods. Therefore, build-up welding is easy even in the case of hardly workable material.

The process for producing hydrocarbons according to the present invention will be described. The process for producing hydrocarbons of the present invention comprises a thermal cracking process, a decoking process, and a furnace tube replacement process.

The thermal cracking process comprises heating a furnace tube composed of a multi-layered heat resistant metal tube comprising a substrate tube composed of a heat resistant metal and a surface layer composed of a Cr—Ni alloy, which is formed on the inner surface of the substrate tube, to a predetermined temperature; and supplying a hydrocarbon raw material gas into the furnace tube to thermally decompose the hydrocarbon raw material gas.

Composition of cracked gas obtained by thermal cracking of the hydrocarbon raw material gas generally depends on the decomposition temperature. In the case that naphtha is thermally cracked to produce ethylene, for example, the yield of ethylene is maximized at a decomposition temperature of about 1000° C. On the other hand, the heat resistance of the furnace tube is restricted by the material of the substrate tube. Accordingly, as the decomposition temperature, it is preferable to select an optimum temperature, considering them. In the production of ethylene, for example, the decomposition temperature is preferably in the range of 800 to 920° C. in the case of using a multi-layered heat resistant metal tube comprising a substrate tube composed of heat resistant cast steel such as HP material as the furnace tube.

No coke is accumulated on the inner surface of the furnace tube in the initial stage of thermal cracking, therefore, a high decomposition temperature can be obtained even with a relatively low surface temperature of the furnace tube (tube internal temperature, hereinafter referred to as “TMT”). On the other hand, coke is accumulated on the inner surface of the furnace tube in accordance with continuation of the operation to deteriorate the heat conductivity. In order to keep the decomposition temperature constant, to raise TMT is needed. In an actual furnace, temperature control of the furnace tube is performed by use of TMT or coil outlet temperature (hereinafter referred to as “COT”) so as to keep the decomposition temperature at a predetermined temperature.

In control of the decomposition temperature by use of TMT, an optimum temperature is selected as the TMT just after starting of thermal cracking (hereinafter referred to as “an initial TMT”), considering the intended composition of cracked gas, the material of substrate tube, the production cost of cracked gas, and the like. In the case that the decomposition temperature is constant, in general, the raw material input to the furnace tube can be increased in accordance with rise of an initial TMT.

On the other hand, in general, the higher the initial TMT is, the more the carburization is accelerated. This shortens the life of the furnace tube. However, in the present invention, the multi-layered heat resistant metal tube is used as the furnace tube, therefore, even if the initial TMT is raised, deterioration by carburization can be remarkably inhibited, compared with a furnace tube composed of only the substrate tube.

To obtain high production efficiency in the use of the multi-layered heat resistant metal tube having the substrate tube composed of heat resistant cast steel, the initial TMT is preferably set to 940° C. or more, further preferably 960° C. or more, and furthermore preferably 980° C. or more. However, an excessively high initial TMT temperature is not preferable because coking becomes excessive, or carburization is accelerated. Accordingly, as the initial TMT, it is preferable to select an optimum temperature which is not higher than the heat resistant limit of the multi-layered heat resistant metal tube so that the highest production efficiency can be obtained.

In the case that the thermal cracking starts at a certain initial TMT, the operation is continued while gradually raising the TMT, and interrupted when the TMT reaches a certain limit value (hereinafter referred to as “an upper limit TMT”). In general, the higher the upper limit TMT is, the more the run-length to decoking can be extended, the larger the raw material input can be made, or the more the decomposition temperature can be raised. However, to make the upper limit higher accelerates the carburization, thereby shortening the life of the furnace tube.

However, in the present invention, the multi-layered heat resistant metal tube is used as the furnace tube, therefore, the upper limit TMT can be raised without significantly deteriorating the life of the furnace tube. To obtain the high production efficiency in the use of the multi-layered heat resistant metal tube with the substrate tube of heat resistant cast steel as the furnace tube, concretely, the upper limit TMT is preferably 1120° C. or more, further preferably 1130° C. or more, more preferably 1140° C. or more, and furthermore preferably 1150° C. or more. An excessively high upper limit TMT should be avoided because the furnace tube is creep-deformed, or carburization is accelerated. Accordingly, as the upper limit TMT, it is preferable to select an optimum temperature which is not higher than the heat resistant limit of the multi-layered heat resistant metal tube so that the highest production efficiency can be obtained.

The raw material input is selected in an optimum manner according to the initial TMT, the upper limit TMT, an intended decomposition temperature, the heat resistant limit of the multi-layered heat resistant tube, the production efficiency, and the like. In general, if the decomposition temperature is constant, the higher the initial TMT is, the more the raw material input can be increased. When the initial TMT and the upper limit TMT are constant, the smaller the raw material input is, the higher the decomposition temperature can be kept.

The time up to decoking from the start of thermal cracking (hereinafter referred to as “a run-length”) is selected in an optimum manner according to the initial TMT, the upper limit TMT, the raw material input, the heat resistant limit of the multi-layered heat resistant metal tube, the production efficiency and the like. In general, when the thermal cracking is performed in a condition hardly causing coking, the run-length can be relatively extended since the ratio of rise of TMT is small. On the other hand, when the thermal cracking is performed in a condition easily occurring coking, the run-length is relatively shortened since the rate of rise of TMT is increased.

In the case that the hydrocarbon raw material gas is thermally cracked, two types of catalytic coke and thermal coke are generally generated. In the present invention, a multi-layered heat resistant metal tube with a predetermined surface layer is used as the furnace tube. Therefore, generation of catalytic coke is inhibited. In a relatively gentle thermal cracking condition, coking is hardly caused. Accordingly, the run-length can be extended, compared with a furnace tube composed of only the substrate tube.

On the other hand, in a relatively severe thermal cracking condition, the amount of generated-thermal coke is increased, therefore, the effect of extending the run-length is minimized. However, since the carburization is inhibited by the surface layer, the life of the furnace tube can be largely increased, compared with the furnace tube composed of only the substrate tube, even in thermal cracking under a sever condition.

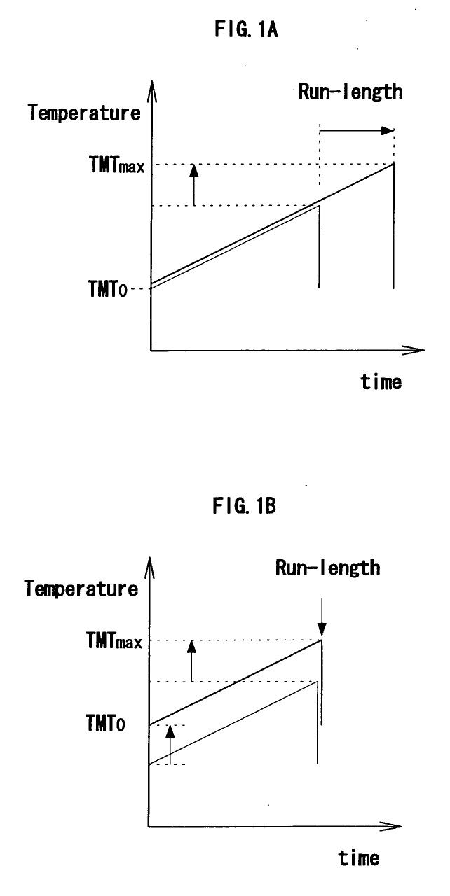

FIGS. 1A and 1B show the relation between TMT and run-length (R/L). In the present invention, the upper limit TMT can be raised without significantly shortening the life of the furnace tube, therefore, the production efficiency can be significantly improved.

Namely, as shown in FIG. 1A, in the case that the initial TMT (TMT₀) and the raw material input are constant, the run-length (R/L) can be extended by raising the upper limit TMT (TMTₘₐₓ).

Concretely, by raising the upper limit TMT, the run-length can be made 1.3 times or more the case of performing thermal cracking at the same initial TMT (or decomposition temperature) by use of a furnace tube composed of only the substrate tube. Further, if the thermal cracking condition is optimized, the run-length can be made 1.8 times or more the case of using the furnace tube composed of only the substrate tube.

As shown in FIG. 1B, in the case that the run-length (R/L) is set to be the same, the raw material input can be increased by raising the upper limit TMT (TMTₘₐₓ) (and the initial TMT (TMT₀)).

Concretely, by raising the upper limit TMT, the raw material input can be made 1.05 times or more the case of performing thermal cracking in the same run-length by use of a furnace tube composed of only the substrate tube.

In this case, the life of the furnace tube is shortened according to the rise of the upper limit TMT. However, in both cases, the life of the furnace tube is 2 times or more the case of using the furnace tube composed of only the substrate tube, or 3 times or more depending on the thermal cracking condition. By raising the initial TMT and the upper limit TMT, the frequency of decoking can be reduced, or the raw material input can be increased. Accordingly, the production cost can be significantly reduced.

The decoking process is a process for interrupting the operation after the lapse of a predetermined run-length and removing coke accumulated on the inner surface of the furnace tube. The method of removing coke is not particularly limited, and various methods can be employed. In general, the removal is performed by supplying only steam while stopping the supply of the hydrocarbon raw material gas to the furnace tube.

The furnace tube replacement process is a process for performing a replacement of furnace tube after the lapse of a predetermined operation time (the time up to the replacement of furnace tube from the start of thermal cracking).

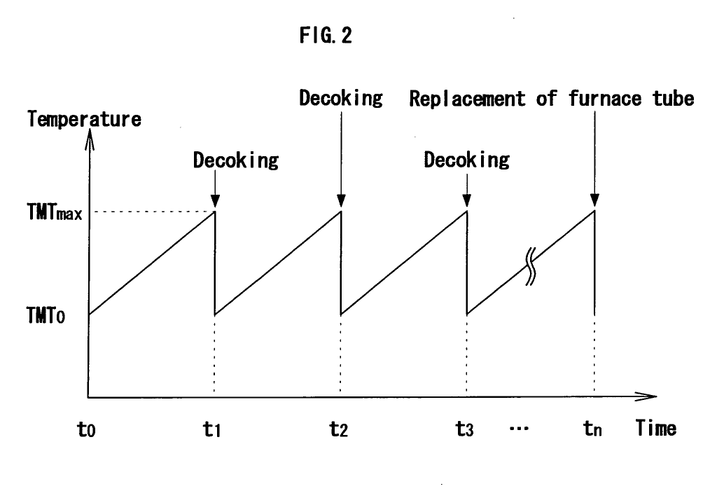

FIG. 2 shows the relation between TMT and time in thermal cracking. In an actual furnace, as shown in FIG. 2, the thermal cracking is started at a predetermined initial TMT (TMT₀), and the operation is continued until TMT reaches a predetermined upper limit TMT (TMTₘₐₓ). When TMT reaches the upper limit TMT (TMTₘₐₓ) after the lapse of a predetermined run-length(=t₁−t₀), the operation is interrupted to perform decoking.

After decoking, the thermal cracking is restarted at a predetermined TMT (TMT₀). When TMT reaches the upper limit TMT (TMTₘₐₓ) after the lapse of a predetermined run-length(=t₂−t₁), second decoking is carried out. The continuous operation at predetermined run-length(=t_{k|1}−tₖ) and the decoking are subsequently repeated in the same manner. When the operation time(=tₙ−t₀) reaches the life of the furnace tube, then the furnace tube replacement is carried out.

The “life of the furnace tube” is defined as the sum (A+B) of the life of the surface layer (A) and the life of the substrate tube (B). The “life of the surface layer” is defined as the time until the diffusion of carbon starts from the surface layer to the substrate tube. Further, the “life of the substrate tube” is defined as the time when the carbon content of the substrate tube exceeds a certain fixed value by carburization.

In the case of the furnace tube composed of only the substrate tube, for example, carbon is diffused from the inner surface of the furnace tube according to the thermal cracking of the hydrocarbon raw material gas, and when the carbon content exceeds a certain fixed value, graphite is deposited. Since the deposited graphite may be a starting point of breakage, it is necessary to replace the furnace tube at the latest before the graphite is deposited on the whole section of the furnace in order to avoid a breakage accident.

The carbon content causing the deposition of graphite is varied depending on the material of the furnace tube. For example, in the case of HP—Nb material that is a kind of heat resistant cast steel, the carbon content starting deposition of graphite is about 5 wt %. In the case that thermal cracking of the hydrocarbon raw material gas is performed by use of the furnace tube composed of only the HP—Nb material, the time when the carbon content on the inner surface side of the furnace tube reaches about 5% is about 3 to 5 years in a relatively gentle thermal cracking condition (concretely, in the case that an upper limit TMT is lower than 1100° C.), although it is varied depending on the thermal cracking condition.

In the case that the multi-layered heat resistant metal tube is used as the furnace tube, the carbon diffused from the inner surface of the furnace tube is trapped first by the surface layer. In the case of the Cr—Ni alloy having the abovementioned composition, the carbon content starting the deposition of graphite is about 7 wt %, which is larger than in the substrate tube. Further, in the case of the multi-layer heat resistant metal tube, its strength is based on the substrate tube. Therefore, even if graphite is deposited on the whole section of the surface layer, it is not the starting point of breakage. Further, the multi-layered heat resistant metal tube is also characterized by that the diffusion of carbon to the substrate tube is inhibited until the carbon content on the surface layer exceeds a certain fixed value.

The diffusion of carbon to the substrate tube starts at the time when the carbon content on the interface side of the surface layer reaches about 3 w %, although the time is slightly varied depending on the composition of the surface layer. This corresponds to about 0.03 wt % in terms of the carbon content solid-solved in the interface-side matrix of the surface layer. These points are found first by the present inventors.

In the case of the surface layer composed of the Cr—Ni alloy, the time until diffusion of carbon to the substrate tube starts (namely, the life of the surface layer) is concretely 1 time or more the life of the substrate tube in the case of performing thermal cracking under the same condition. The life of the surface layer is twice or more the life of the substrate tube, or 2.5 times or more depending on the thermal cracking condition or the like.

Consequently, the life of the furnace tube in the use of the multi-layered heat resistant metal tube is extended more than the life of the furnace tube in the case of performing thermal cracking in the same condition by use of the furnace tube composed of only the substrate tube. Concretely, the life of the multi-layered heat resistant metal tube is twice or more the life of the furnace tube composed of only the substrate tube, or 3 times or more depending on the thermal cracking condition or the like.

The carburization quantity of the furnace tube depends on the exposing temperature and time of the furnace tube. The actual carburization quantity in actual furnace can be estimated by time temperature parameter (TTP) method based on the accelerated carburetion test data. The Larson-Miller parameter (LMP) that is one kind of TTP methods can be represented by the following equation (a).
LMP=(273+T)(20+log t) (a)
wherein T is temperature (° C.), and t is time (hr)

The actual carburization quantity is varied depending on carburization conditions. The relation between temperature and time (t₁) until the carbon content of the surface layer composed of Cr—Ni alloy having the abovementioned composition reaches about 3 wt % can be represented, by using LMP, by the following equation (b)
(273+TMTₘ)(20+log t₁)≧32.5×10³ (b)
wherein TMTₘ is the average tube internal temperature (the average TMT), and t₁ is the operation time (hr).

Based on the equation (b), the life (t) of the furnace tube with the surface layer composed of Cr—Ni alloy can be represented by the following equation (1).
t≧t₁ (1)
wherein t₁=exp{[32.5×10³/(273+TMTₘ)−20]×1n10}, and TMTₘ is the average tube internal temperature (° C.).

The equation (1) means that the time when the carbon content of the surface layer reaches about 3 wt % is, for example,
(a) about 15.6 years with an average TMT of 1020° C. in thermal cracking;
(b) about 10.0 years with an average TMT of 1030° C. in thermal cracking;
(c) about 6.5 years with an average TMT of 1040° C. in thermal cracking;
(d) about 4.2 years with an average TMT of 1050° C. in thermal cracking;
(e) about 2.8 years with an average TMT of 1060° C. in thermal cracking; and
(f) about 1.8 years with an average TMT of 1070° C. in thermal cracking.

In every case, since the carburization to the substrate tube hardly progresses in the condition of the equation (1), the life of the furnace tube corresponds to the abovementioned years plus the life of the substrate tube.

The relation between temperature and time (t₂) until the carbon content on the inner surface side of the substrate tube composed of heat resistant cast steel reaches about 5 wt % can be represented by the following equation (c).
(273+TMTₘ)(20+log t₂)≧31.9×10³ (c)
wherein TMTₘ is the average tube internal temperature (the average TMT), and t₂ is the operation time (hr).

From the equations (b) and (c), the life (t) of the furnace tube with the surface layer composed of Cr—Ni alloy, which is formed on the inner surface of the substrate tube composed of heat resistant cast steel, can be represented by the following equation (2).
t≧t₁+t₂ (2)
wherein t₁=exp{[32.5×10³/(273+TMTₘ)−20]×1n10}, t₂=exp{[31.9×10³/(273+TMTₘ)−20]×1n10}, and TMTₘ is the average tube internal temperature (° C.)

The equation (2) means that the time when the carbon content of the surface layer reaches about 3 wt %, and the carbon content on the inner surface side of the substrate tube reaches about 5 wt % is, for example,
(a) about 21.0 years with an average TMT of 1020° C. in thermal cracking;
(b) about 13.5 years with an average TMT of 1030° C. in thermal cracking;
(c) about 8.7 years with an average TMT of 1040° C. in thermal cracking;
(d) about 5.7 years with an average TMT of 1050° C. in thermal cracking;
(e) about 3.7 years with an average TMT of 1060° C. in thermal cracking; and
(f) about 2.5 years with an average TMT of 1070° C. in thermal cracking.

In every case, since the graphite starts to deposit on the inner surface side of the substrate tube in the condition of the equation (2), the furnace tube replacement is preferably carried out as soon as possible.

The relation of the life of the furnace tube having the surface layer of Ni—Cr alloy formed on the inner surface of the substrate tube of heat resistant cast steel with an initial TMT, an upper limit TMT and an average TMT is shown in Table 1.

TABLE 1

| | Upper | | Life (years) | | |
|---|---|---|---|---|---|
| Initial | limit TMT | Average | Surface | Substrate | |
| TMT (° C.) | (° C.) | TMT (° C.) | layer | tube | Total |
| 940 | 1100 | 1020 | ≧15 | ≧5 | ≧20 |
| | 1110 | 1025 | ≧12 | ≧4 | ≧16 |
| | 1120 | 1030 | ≧10 | ≧3 | ≧13 |
| | 1130 | 1035 | ≧8 | ≧2 | ≧10 |
| | 1140 | 1040 | ≧6 | ≧2 | ≧8 |
| | 1150 | 1045 | ≧5 | ≧1 | ≧6 |
| 950 | 1100 | 1025 | ≧12 | ≧4 | ≧16 |
| | 1110 | 1030 | ≧10 | ≧3 | ≧13 |
| | 1120 | 1035 | ≧8 | ≧2 | ≧10 |
| | 1130 | 1040 | ≧6 | ≧2 | ≧8 |
| | 1140 | 1045 | ≧5 | ≧1 | ≧6 |
| | 1150 | 1050 | ≧4 | ≧1 | ≧5 |
| 960 | 1100 | 1030 | ≧10 | ≧3 | ≧13 |
| | 1110 | 1035 | ≧8 | ≧2 | ≧10 |
| | 1120 | 1040 | ≧6 | ≧2 | ≧8 |
| | 1130 | 1045 | ≧5 | ≧1 | ≧6 |
| | 1140 | 1050 | ≧4 | ≧1 | ≧5 |
| | 1150 | 1055 | ≧3 | ≧1 | ≧4 |
| 970 | 1100 | 1035 | ≧8 | ≧2 | ≧10 |
| | 1110 | 1040 | ≧6 | ≧2 | ≧8 |
| | 1120 | 1045 | ≧5 | ≧1 | ≧6 |
| | 1130 | 1050 | ≧4 | ≧1 | ≧5 |
| | 1140 | 1055 | ≧3 | ≧1 | ≧4 |
| | 1150 | 1060 | ≧2.5 | ≧0.5 | ≧3 |
| 980 | 1100 | 1040 | ≧6 | ≧2 | ≧8 |
| | 1110 | 1045 | ≧5 | ≧1 | ≧6 |
| | 1120 | 1050 | ≧4 | ≧1 | ≧5 |
| | 1130 | 1055 | ≧3 | ≧1 | ≧4 |
| | 1140 | 1060 | ≧2.5 | ≧0.5 | ≧3 |
| | 1150 | 1065 | ≧2 | ≧0.5 | ≧2.5 |
| 990 | 1100 | 1045 | ≧5 | ≧1 | ≧6 |
| | 1110 | 1050 | ≧4 | ≧1 | ≧5 |
| | 1120 | 1055 | ≧3 | ≧1 | ≧4 |
| | 1130 | 1060 | ≧2.5 | ≧0.5 | ≧3 |
| | 1140 | 1065 | ≧2 | ≧0.5 | ≧2.5 |
| | 1150 | 1070 | ≧1.5 | ≧0.5 | ≧2 |

In the process for producing hydrocarbons of the present invention, since the multi-layered heat resistant metal tube is used as the furnace tube, the oxidation resistance and the carburization resistance are remarkably improved, compared with in the use of a furnace tube composed of only the substrate tube. Therefore, the life of the furnace tube is improved twice or more the life in the case of performing thermal cracking in the same condition by use of the furnace tube composed of only the substrate tube, or 3 times or more depending on the thermal cracking condition.

Even in the multi-layered heat resistant metal tube, when the upper limit TMT (namely, the average TMT) is raised, the life of the furnace tube is reduced because carburization is accelerated. However, the multi-layered heat resistant tube is excellent in carburization resistance. Therefore, if the upper limit TMT is raised, then the rate of deterioration of the furnace tube life is remarkably minimized, compared with the furnace tube composed of only the substrate tube. Therefore, the use of the process according to the present invention enables a reduction in the frequency of decoking, an increase in the raw material input, or a decomposition temperature closer to an ideal temperature. Further, according to this, the production cost of hydrocarbons such as ethylene can be significantly reduced.

### EXAMPLES

### Example 1

A surface layer (PPW layer) composed of 44.5% Cr—Ni alloy was build-up welded on the inner surface of a centrifugal casting steel pipe (substrate tube) composed of a HP—Nb material by means of plasma powder welding (PPW) to produce a multi-layered heat resistant metal tube (PTT). The substrate tube has an outer diameter of 80.1 mm, an inner diameter of 63.5 mm, and a surface layer thickness of 2 mm. Using the resulting multi-layered heat resistant metal tube as a furnace tube, the accelerated carburetion test and thermal cracking of naphtha in actual furnace were carried out.

### Comparative Example 1

Using a centrifugal casting steel pipe composed of HP—Nb material as a furnace tube, the accelerated carburetion test and the thermal cracking of naphtha in actual furnace were carried out. The furnace tube has an outer diameter of 80.1 mm and an inner diameter of 63.5 mm.

(Evaluation)

After the accelerated carburetion test and the thermal cracking for a fixed period in actual furnace were ended, the carbon contents on the inner surface and outer surface of each tube were measured by use of a carburization meter.

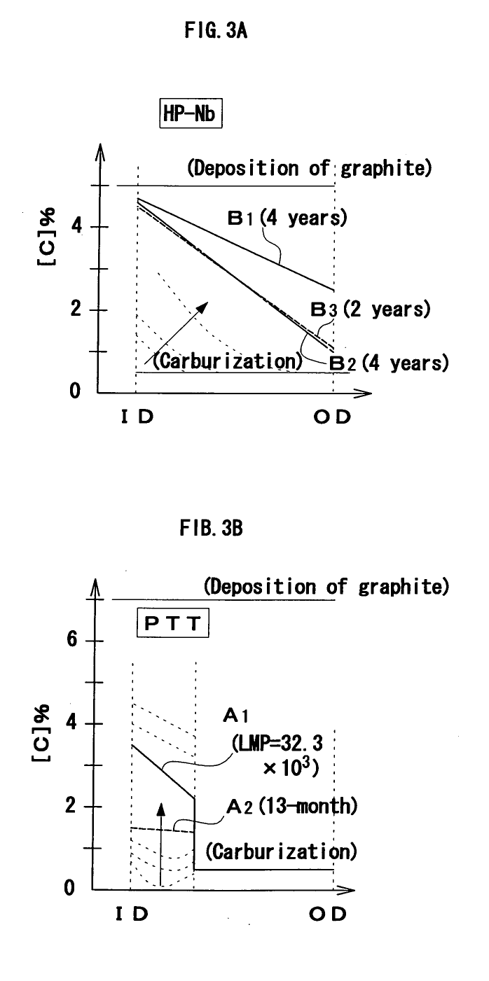

FIG. 3A shows the carbon content of the furnace tube in the thermal cracking of naphtha in actual furnace by use of the furnace tube composed of the HP—Nb material. Since the carburization progresses from the inner surface of the furnace tube, the carbon content on the inner diameter (ID) side of the furnace tube is larger than that on the outer diameter (OD) side. The carburization rate is varied depending on the thermal cracking condition. Therefore, the carbon content of the inner diameter-side reaches about 4.5 wt % in about 4 years under conditions B₁ and B₂, and in about 2 years under condition B₃. Since graphite is deposited with a carbon content exceeding about 5 wt % in the case of the HP—Nb material, the life of the HP—Nb material is found to be about 3 to 5 years.

FIG. 3B shows the carbon content in an accelerated carburetion test by use of a multi-layered heat resistant metal tube (PTT) formed by PPW (condition A₁) and in thermal cracking in actual furnace (condition A₂). As is apparent from FIG. 3B, the carbon content of the PPW layer after 13-month thermal cracking in actual furnace is about 1.5 wt %, which shows no diffusion of carbon to the substrate tube. In the accelerated carburetion test in the condition of LMP=32.3×10³, the carburization quantity on the inner diameter (ID) side is about 3.5%. The carbon content on the interface side is about 2%, which shows no diffusion of coke to the substrate tube.

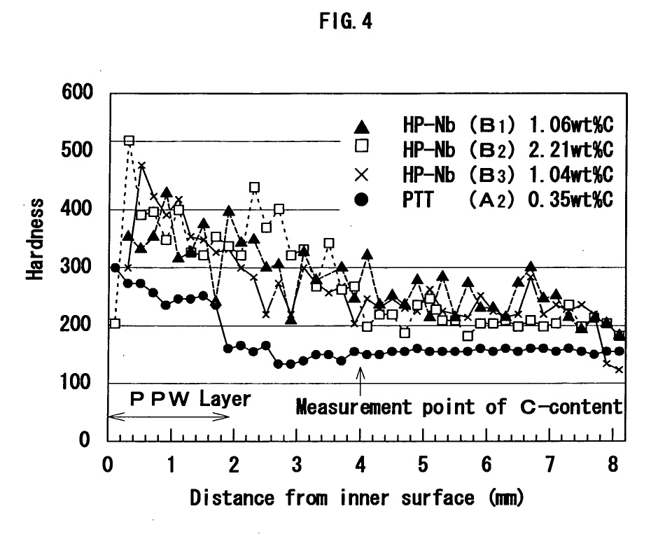

The PTT after the thermal cracking in actual furnace (condition A₂) and the NP—Nb material after the thermal cracking in actual furnace (conditions B₁ to B₃) were cut vertically to the tube axes, and the hardness of each section was measured. FIG. 4 shows the results.

It is found from FIG. 4 that, in the use of the furnace tube composed of HP—Nb material, the hardness increases more toward the inner surface of the furnace tube. This reason is that carbon is diffused from the inner surface of the furnace tube toward the outer surface of the furnace tube. On the other hand, in the case of the PTT, the hardness of the PPW layer is increased, however, the hardness of the substrate tube is low, compared with those in the conditions B₁ to B₃. This reason is that the PPW layer captures the carbon diffused from the inner surface of the furnace tube to inhibit the diffusion of the carbon to the substrate tube.

For these furnace tubes, the carbon content in a point about 4 mm from the inner surface of each furnace tube was measured. Consequently, in the case of the HP—Nb material, the quantity was 1.06 wt % in the condition B₁, 2.21 wt % in the condition B₂, and 1.04 wt % in the condition B₃. In contrast to this, in the condition A₁ using PTT, the carbon content was 0.35 wt %, which was substantially equal to the carbon content before thermal cracking.

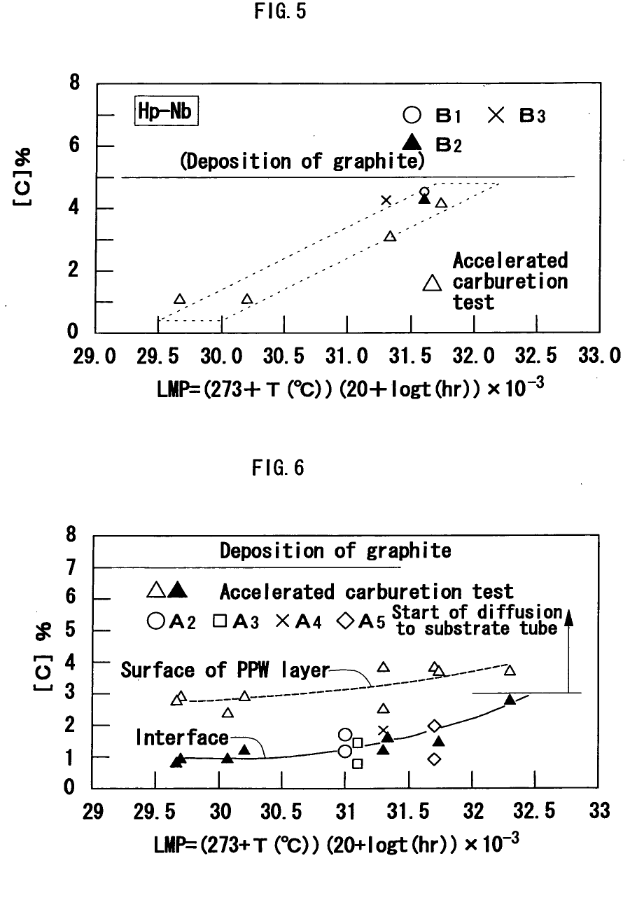

FIG. 5 shows the relation between the LMP of the furnace tube composed of HP—Nb material and the carbon content on the inner surface side of the furnace tube. FIG. 6 shows the relation between the LMP of the furnace tube composed of PTT and the carbon contents on the surface side and interface side of the PPW layer.

In the case of the HP—Nb material, as shown in FIG. 5, a satisfactory correlation was recognized between the LMP and the carbon content even if the thermal cracking conditions in actual furnace (conditions B₁, B₂, and B₃) were slightly differed. In the case that LMP is 31.9×10³, the carbon content is about 5%, which gets close to the limit carbon content starting the deposition of graphite in the furnace tube. This corresponds to, for example, about 5 years with an average TMT of 1020° C.

In the case of the PTT, similarly, a satisfactory correlation was recognized between the LMP and the carbon content, as shown in FIG. 6, even if the thermal cracking conditions in actual furnace (conditions A₂, A₃, A₄, and A₅) were slightly differed. When LMP is 32.5×10³, the carbon content on the interface side of the PPW layer reaches about 3%. This corresponds to, for example, about 16 years with an average TMT of 1020° C. Namely, in the same thermal cracking condition, the life of the PTT is about 4 times that of the HP—Nb material.

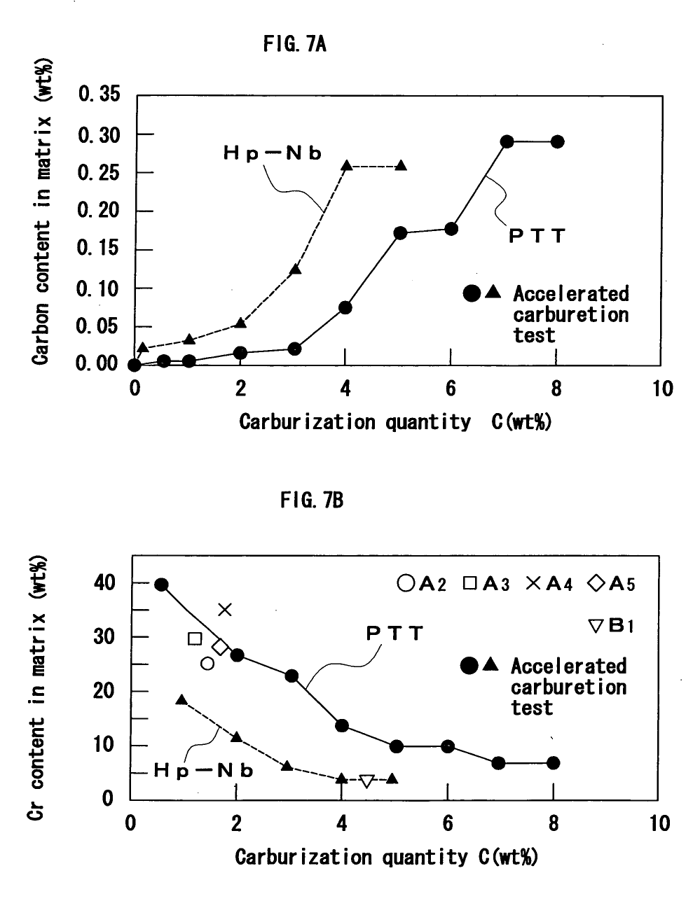

FIG. 7A shows the relation between carburization quantity and C content in matrix after the accelerated carburetion test, which were measured for PTT and HP—Nb material. FIG. 7B shows the relation between carburization quantity and Cr content in matrix after the accelerated carburetion test and after the thermal cracking in actual furnace (PTT: conditions A₂ toA₅, HP—Nb material: condition B₁), which were measured for the PTT and HP—Nb material. In each case, the value of interface-side in the PPW layer is taken as data for the PTT.

As is apparent from FIGS. 7A and 7B, in the case of the NP—Nb material, the Cr content in matrix decreases, and the carbon content in matrix increases, in accordance with the increase in carburization quantity. This shows that part of the carbon diffused from the inner surface of the furnace tube is consumed for generation of chromium carbide, and part of the rest continuously diffuses toward the outer surface side.

In the case of the PTT, the Cr content in matrix of the PPW layer suddenly decreases up to a carburization quantity of about 3 wt % (about 0.03 wt % by the carbon content in matrix), while the C content in matrix of the PPW layer hardly increases. This shows that most of the carbon diffused from the inner surface of PTT is consumed for generation of chromium carbide, and the PPW layer inhibits the diffusion of carbon to the substrate tube. On the other hand, when the carburization quantity of the PPW layer exceeds 3 wt %, the carbon content in matrix of the PPW layer suddenly increases over the carbon content in matrix of the substrate tube. This shows that when the carburization quantity of the PPW layer exceeds about 3 wt %, the diffusion of carbon to the substrate tube starts.

Further, measurement of the thickness of a Cr-deficient layer on the inner surface of the furnace tube after thermal cracking in actual furnace and analysis of the component thereof were performed. The results are shown in Table 2. In actual furnace, the inner surface of the furnace tube is exposed to an oxidizing atmosphere at the time of decoking, therefore, the Cr-deficient layer is formed on the inner surface of the furnace tube by oxidation. The Cr-deficient layer formed on the inner surface of the PTT by 13-month operation was only 0.05 mm thick, against a thickness of 0.15 to 0.45 mm of the Cr-deficient layer formed on the inner surface of the HP—Nb material by operation for 2 to 4 years. This shows that the thickness of the Cr-deficient layer formed on the inner surface of the PTT is equal to or less than the HP—Nb in terms of the same operation time (namely, the oxidation resistance of PTT is equal to or more than that of the HP—Nb).

TABLE 2

| | | Thickness of | Composition of Cr-deficient | |
|---|---|---|---|---|
| | Operating | Cr-deficient | layer | |
| Furnace tube | condition | layer (mm) | Cr (wt %) | Ni (wt %) |
| HP-Nb | B₁ | 0.45 | 11.5 | 40.0 |
| | B₂ | 0.40 | 15.0 | 32.0 |
| | B₃ | 0.15 | 12.5 | 27.0 |
| PTT | A₂ | 0.05 | 27.0 | 61.6 |

The embodiments of the present invention have been described above in detail. The present invention is not limited by the above embodiments, and various modifications can be made within the range not departing from the gist of the present invention.

Although a centrifugal casting steel pipe composed of heat resistant cast steel was used as the substrate tube in the above examples, for example, the same can be said in use of substrate tubes composed of other heat resistant metals such as stainless steel and Ni-based cemented carbide. Namely, by forming a surface layer composed of a Cr—Ni alloy on the inner surface, the life of the furnace tube can be made twice or more the life of a furnace tube composed of only the substrate tube, or 3 times or more depending on the thermal cracking condition. The carburization resistance are improved, therefore, the upper limit TMT can be increased without significantly deteriorating the life.

The process for producing hydrocarbons of the present invention can be applied to generation processes and production processes of various gases where heat resistance and carburization resistance of furnace tubes are required, in addition to the process for thermally cracking naphtha to produce ethylene, propylene and the like.

1. A process for producing hydrocarbons, comprising:
a thermal cracking process for heating a furnace tube composed of a multi-layered heat resistant metal tube comprising a substrate tube composed of a heat resistant metal and a surface layer of a Cr—Ni alloy formed on an inner surface of said substrate tube to a predetermined temperature, and supplying a hydrocarbon raw material gas into said furnace tube to thermally decompose the hydrocarbon raw material gas;
a decoking process for removing coke accumulated on the inner surface of said furnace tube; and
a furnace tube replacement process for performing replacement of said furnace tube after the lapse of an operation time longer than the life of said substrate tube in the case of performing said thermal cracking under the same condition by use of a furnace tube composed of only said substrate tube.

2. The process for producing hydrocarbons according to claim 1, wherein said substrate tube is a centrifugal casting steel pipe composed of a heat resistant cast steel.

3. The process for producing hydrocarbons according to claim 1, wherein said Cr—Ni alloy includes Cr: 36 to 49 wt % and Ni: 35 to 63 wt %.

4. The process for producing hydrocarbons according to claim 2, wherein said Cr—Ni alloy includes Cr: 36 to 49 wt % and Ni: 35 to 63 wt %.

5. The process for producing hydrocarbons according to any one of claims 1 to 4, wherein said furnace tube replacement process comprises replacing said furnace tube after the lapse of an operation time two times or more the life of said substrate tube.

6. The process for producing hydrocarbons according to any one of claims 1 to 4, wherein said furnace tube replacement process comprises replacing said furnace tube when the operation time (t (hr)) satisfies the relation shown by the following equation (1):
t≧t₁ (1)
wherein t₁=exp{[32.5×10³/(273+TMTₘ)−20]×1n10}, and TMTₘ is the average tube internal temperature (° C.).

7. The process for producing hydrocarbons according to any one of claims 1 to 4, wherein said furnace tube replacement process comprises replacing said furnace tube when the operation time (t (hr)) satisfies the relation shown by the following equation (2):
t≧t₁+t₂ (2)
wherein t₁=exp{[32.5×10³/(273+TMTₘ)−20]×1n10}, t₂=exp{[31.9×10³/(273+TMTₘ)−20]×1n10}, and TMTₘ is the average tube internal temperature (° C.).

8. The process for producing hydrocarbons according to any one of claims 1 to 4, wherein said furnace tube replacement process comprises replacing said furnace tube when the operation time reaches 13 years or more under the condition of an average tube internal temperature of 1030° C. or less.

9. The process for producing hydrocarbons according to any one of claims 1 to 4, wherein said furnace tube replacement process comprises replacing said furnace tube when the operation time reaches 5 years or more under the condition of an average tube internal temperature of 1030° C. or more and 1050° C. or less.

10. The process for producing hydrocarbons according to any one of claims 1 to 4, wherein said furnace tube replacement process comprises replacing said furnace tube when the operation time reaches 2 years or more under the condition of an average tube internal temperature of 1050° C. or more and 1070° C. or less.

11. The process for producing hydrocarbons according to any one of claims 1 to 4, wherein said decoking process comprises removing coke accumulated on the inner surface of said furnace tube when the tube internal temperature reaches 1120° C. or more.

12. The process for producing hydrocarbons according to any one of claims 1 to 4, wherein said decoking process comprises removing coke accumulated on the inner surface of said furnace tube when the tube internal temperature reaches 1140° C. or more.
